(19) United States
(12) Patent Application Publication
(10) Pub. No.: US 2012/0123304 A1
(43) Pub. Date: May 17, 2012

(54) MOVING STANDING WAVES

(75) Inventor: Andrey Rybyanets, Rostov on Don (RU)

(73) Assignee: WAVOMED LTD., Tel-Aviv (IL)

(21) Appl. No.: 13/377,561

(22) PCT Filed: Jun. 12, 2010
(86) PCT No.: PCT/IB10/52624

§ 371 Date: Dec. 11, 2011

Related U.S. Application Data
(60) Provisional application No. 61/187,407, filed on Jun. 16, 2009.

(51) Int. Cl. A61N 7/00

(52) U.S. Cl. 601/2

(57) ABSTRACT

A method of treating a region of tissue with ultrasound, the method comprising: generating a first ultrasound standing wave (USW) pattern at a first frequency in the region; and simultaneously generating a second USW pattern in the region spatially overlapping the first USW pattern at a second frequency different from the first frequency.

### CROSS REFERENCE TO RELATED APPLICATIONS

This application claims the benefit of priority from U.S. provisional patent application No. 61/187,407 filed Jun. 16, 2009, which is incorporated herein by reference in its entirety.

### BACKGROUND

Various methods are known for delivering and coupling acoustic energy to a region of tissue to perform a diagnostic and/or therapeutic and/or cosmetic procedure on a patient's tissue. Among such procedures are for example, non-invasive assaying of blood analytes, drug delivery by phonophoresis, lithotripsy, tissue ablation and lysis of fat cells for cosmetic removal of adipose tissue.

For many types of therapeutic and/or cosmetic acoustic applications, such as for example lithotripsy, tissue ablation and lysis noted above, sufficient acoustic energy must be delivered to a tissue region to destroy and remove tissue in the region. Generally, the acoustic energy is delivered by focusing at least one beam of relatively intense ultrasound on the region. The high intensity, focused ultrasound, conventionally referred to by the acronym “HIFU”, may be used to generate various thermal and mechanical effects on tissue that include local heating of tissue and/or cavitation that disrupts and destroys the tissue. Tissue raised to and maintained at a temperature above about 42° C. rapidly dies and mechanical stresses generated by cavitation breach and tear cell membranes of the tissue.

However, it is often difficult to efficiently treat relatively large volumes of tissue using HIFU. For example, HIFU beams are often focused to relatively small volumes of tissue and can require relatively large dwell times at the focal volumes to destroy tissue therein. Typically, a focal volume of a HIFU beam is substantially contained within a prolate ellipsoid. For a frequency of ultrasound equal to about 200 kHz, which is commonly used in ultrasound tissue treatment, the ellipsoid has a long axis of about 15 mm along a direction of propagation of the beam and a maximum cross section perpendicular to the propagation direction having a diameter of about 7.5 mm. For frequency of about 1 MHz, the long axis is about 3 mm and the cross section has a diameter of about 1.5 mm. In general, the focal volume has lateral diameter of approximately 1 wavelength and a length of between about 2-3 wavelengths. (Boundaries of the focal volume are assumed to be in regions where acoustic intensity is attenuated by about 6 dB.) Treating an extended region of tissue with HIFU, for example to lyse adipose tissue, can therefore often be a relatively tedious task that requires a relatively long time to perform. As a result, various techniques have been proposed and/or used for expanding a useful focal volume of HIFU beams and for electronically and/or mechanically scanning the beams to treat relatively large tissue volumes.

However, controlling HIFU beams to deliver effective acoustic energy that is spatially relatively homogenous over an extended tissue volume that is a desired target for treatment and that does not adversely affect non-target tissue can be problematic. Often configurations of extended focal volume HIFU beams exhibit “hot spots” that limit therapeutic and/or cosmetic use of the beams. And, ultrasound that is propagated into the body so that it is substantially focused in a desired region, generally propagates through and past the focal region and is incident on organs and/or body features for which the ultrasound is not intended.

For example, adipose tissue generally resides in the subcutaneous layer of the skin and is located in a region from about a few mm to a few tens of mm below the skin surface. In procedures for tissue ablation and lysis of fat cells for cosmetic removal of adipose tissue, ultrasound focused to fat tissue below the skin may propagate beyond the adipose tissue, impinge on, and damage internal organs and body features lying below the subcutaneous layer. If the ultrasound is being used to treat belly fat, the ultrasound may for example, be incident on the liver. If the ultrasound is used to treat cellulites in the hip region, the ultrasound may be incident on and reflected from bone tissue below the skin. The reflected ultrasound can interfere with the ultrasound propagated into the body to treat the cellulites and generate a standing acoustic wave having intensity at or near the skin surface that can damage the skin.

F. L. Lizzi et al in an article entitled “Asymmetric Focussed Arrays for Ultrasonic Tumor Therapy” describe using “spherical cap transducers with segmented rectangular electrodes” to provide HIFU beams useable to produce lesions with elliptical cross-sections. By phasing excitation of pairs of rectangular electrodes, undesired axial regions of high acoustic intensity of the beams in planes other than a focal plane of the transducers were suppressed. To treat extended tissue regions, the beams are intended for scanning along a direction substantially perpendicular to the long axes of the elliptical lesions.

US Patent Application Publication US 2005/0154314 to J. U. Quistgaard, entitled “Component Ultrasound Transducer” promulgates objects of the invention of the application as: “to provide a transducer capable of transmitting high intensity ultrasound energy into two or more focal zones simultaneously” and also “to provide for a transducer capable of focusing two or more different frequencies into a single focal zone, or into a group of focal zones.”

U.S. Pat. No. 6,506,171 S. Vitek and N Brenner describe a focused ultrasound system that “includes a plurality of transducer elements disposed about and having an angular position with a central axis”. The various sector elements are excited with phases so that “a first on-axis focal zone and a second off-axis focal zone are created”. The figures in the application show that the second off-axis focal zone is characterized by a plurality of focal regions, in each of which acoustic energy focused to the region has a substantially same spatial energy distribution.

US Patent Application Publication US 2009/0099485 to Sarvazyan et al describes generating an extended ultrasonic standing wave (USW) intensity pattern in a relatively large volume of tissue for “removing significant amounts of adipose tissue from arbitrary body parts”. The use of USW provides for acoustic field strengths at antinodes of the standing wave patterns that are substantially increased relative to conventional HIFU fields.

The extended tissue volume is provided by pinching up and clamping a relatively large volume of tissue to be treated with ultrasound between a pair of ultrasound transducers. The transducers are controlled to radiate ultrasound one towards the other, in opposite directions at a resonant frequency of the clamped tissue, so that they overlap in the clamped tissue and superimpose to generate a USW pattern of nodes and antinodes. Since tissue treatment is located primarily to the antinodal regions of the USW pattern, to attempt to homogenize treatment, the tissue is sequentially treated with USW patterns at different resonant frequencies. Switching the resonant frequencies cause the nodal pattern of standing waves to change its locations. In some embodiments of the invention, the tissue is pinched up between a pair of mechanical clamps. In some embodiments of the invention, instead of being pinched up, tissue to be treated is drawn up into a cup-shaped vessel using a vacuum.

### SUMMARY OF THE INVENTION

An aspect of some embodiments of the invention is related to providing improved methods and apparatus for treating relatively large regions of tissue with ultrasound standing waves (USW). Optionally, the methods and apparatus are used for performing lypolytic, therapeutic, and/or cosmetic treatment of tissue.

An aspect of some embodiments of the invention, relates to providing a method of spatially homogenizing the effects of USW patterns on tissue treated with ultrasound by superimposing USW patterns at least two different frequencies in the tissue. The overlapping of standing wave patterns superpose to generate a pattern of relatively dense spatial distribution of ultrasound nodes and antinodes in the tissue that cyclically shift their location in the tissue at a relatively low frequency equal to about one half a difference in the frequencies of the two USW patterns. The density and cyclical spatial motion of the nodes and antinodes homogenizes the effects of the ultrasound on the tissue.

The inventor has determined that sequentially applying USW patterns to tissue to spatially homogenize ultrasound treatment can be relatively inefficient. Tissue changes, such as cell destruction that results from burning or cavitation, effected in localized, pressure anti-nodal regions of the tissue by a first USW pattern at a first frequency can interfere with the efficacy with which a second USW pattern at a second frequency establishes itself and generates desired changes in the tissue. By simultaneously applying USW patterns at least two different frequencies, in accordance with an embodiment of the invention, desired changes occur spatially homogenized throughout the tissue substantially adiabatically. As a result, all of the at least two USW patterns are relatively efficiently established in the tissue and effective in producing relatively homogenized treatment of the tissue.

An aspect of some embodiments of the invention relates to providing a method of coupling a tissue region to acoustic transducers that are used to provide ultrasound energy to treat the tissue region.

In an embodiment of the invention, the method comprises drawing up a mass of tissue into a vessel using a vacuum and mechanically distorting the drawn up tissue to increase contact area between the tissue and ultrasound transducers in, or mounted to, the walls of the vessel. The increased contact area improves acoustic coupling of the ultrasound transducers to the tissue.

The inventor has determined that with conventional methods of drawing tissue up into a vacuum vessel so that the tissue may be treated with ultrasound, relatively large surface areas of the tissue do not contact ultrasound transducers in, or mounted to, walls of the vessel as a result of the shape that the drawn up tissue acquires in the vessel. By distorting the shape, an increased surface of the drawn up tissue is pressed to the vessel walls. In an embodiment of the invention, distorting the tissue comprises pressing on the tissue with a “shape adapter” that intrudes into the vessel into which the tissue is drawn by the vacuum.

According to an aspect of some embodiment of the invention, mechanically distorting the tissue comprises periodically distorting the tissue. The periodic distortion causes displacement of the tissue relative to a pattern of ultrasound intensity produced by ultrasound transducers that transmit ultrasound into the tissue. The periodic displacement improves spatial homogenization of desired effects of the ultrasound in the tissue. In some embodiments of the invention, the periodic displacement is provided at a frequency close to or substantially equal to a mechanical resonance of the tissue. At resonance, motion of mass points in the tissue relative to the ultrasound field, and thereby homogenization of treatment, tends to be maximized.

An aspect of some embodiments of the invention, relates to providing a method of simultaneously treating a tissue region with ultrasound and an RF electric field. In an embodiment of the invention, acoustic transducers are positioned and excited by time varying RF voltages to generate a pattern of ultrasound in the tissue region so that the same RF voltages generate a time varying RF field that substantially overlaps the ultrasound pattern in the tissue. The RF field pattern preferentially heats relatively high resistance portions of the tissue region. As a result, the configuration of RF and ultrasound fields in a tissue region, in accordance with an embodiment of the invention, can provide synergistic treatment of the tissue region. An ultrasound field and an RF field superposed in accordance with an embodiment of the invention tend to be synergistic in providing therapy to a tissue region and exhibit relatively improved selectivity for tissue to be treated than either field alone.

For example, if the ultrasound field is configured to lyse cells in a portion of the tissue region by cavitation, the resistance of the cavitated portions increases relative to surrounding, non-cavitated tissue. As a result, the RF field preferentially heats, by causing displacements and vibrations of ions in the tissue, the cavitated portions, amplifying the destructive effect of the ultrasound field. The ultrasound thereby tends to focus and localize the RF field effects to the portions to be lysed and provides better discrimination between tissue to be treated and tissue that is not to be treated. By way of another example, it is noted that since both USW and RF fields overlap and are excited at a same frequency or frequencies in a tissue region, the fields operate synergistically in heating the region and generate relatively large amounts of heat in the region, particularly at pressure antinodes of the USW field.

An aspect of some embodiments of the invention relate to providing methods of interfacing ultrasound transducers with a tissue region that provides improved protection for the skin of the tissue region against damage by burning from intense USW fields generated by the transducers.

USW fields generated by an acoustic transducer in a tissue region, typically have a pressure antinode at an interface of the transducer with the tissue. Pressure amplitude of the USW field at the antinode can increase to a relatively very large and skin damaging magnitude. In an embodiment of the invention, protective layers, hereinafter also referred to as “protective buffers”, are formed to couple acoustic transducers with tissue being treated with USW generated by the transducers so that pressure amplitude of the USW along skin surfaces of the tissue at which ultrasound enters the region is relatively small.

In accordance with an embodiment of the invention, the protective buffers have thickness equal to about a quarter wavelength of the ultrasound generated by the transducers. Unlike conventional acoustic couplers used for impedance matching, an ultrasound transducer and a tissue region, which couplers typically have impedance equal to about the square root of the product of the tissue and transducer impedance, the protective buffers, in accordance with an embodiment of the invention, have impedance substantially equal to that of the tissue region. The buffers thereby position relatively low pressure, optionally pressure antinode regions, of the USW field at the skin surface and reduce a tendency of the USW to injure skin surface by burning.

In an embodiment of the invention the protective buffers are formed from a conductive material having an acoustic impedance close to that of biological tissue. For configurations in accordance with an embodiment of the invention, in which electrodes that are used to excite the acoustic transducers are used to generate an RF field in treated tissue, the conductivity aids in establishing the RF field and associated RF currents in the tissue. Various materials, such as types of conductive epoxies, resins or silicones, have acoustic impedances close to that of tissue and may be used for the buffers.

There is therefore provided in accordance with an embodiment of the invention, a method of treating a region of tissue with ultrasound, the method comprising: generating a first ultrasound standing wave (USW) pattern at a first frequency in the region; and simultaneously generating a second USW pattern in the region that spatially overlaps the first USW pattern in the region at a second frequency different from the first frequency.

Optionally, generating the first and second USW patterns comprises generating the patterns so that substantially wherever in the tissue region one of the USW patterns exists it is overlapped by the other pattern.

Additionally or alternatively, generating the first and second USW patterns optionally comprises simultaneously exciting two ultrasound transducers at the first and second frequencies that face each other across the tissue region. Optionally, in the first and second frequencies are resonant frequencies determined by the speed of sound in the tissue region and distance between the transducers. Additionally or alternatively, the method comprises configuring the transducers so that they have resonant frequencies of vibration substantially equal to the first and second resonant frequencies.

In an embodiment of the invention, the method comprises sandwiching a layer of material between a transducer and the skin so that pressure amplitude of the USW along the skin at which ultrasound enters the tissue region is relatively small.

There is further provided in accordance with an embodiment of the invention, a method of treating a region of tissue with ultrasound, the method comprising: exciting an ultrasound transducer to transmit ultrasound through skin to generate an ultrasound standing wave (USW) in the tissue region; and sandwiching a layer of material between the transducer and the skin so that pressure amplitude of the USW along the skin at which ultrasound enters the tissue region is relatively small. Additionally or alternatively, the layer optionally has acoustic impedance substantially equal to that of the tissue region.

In an embodiment of the invention, thickness of the layer is substantially equal to a quarter wavelength of ultrasound that the transducer transmits in the tissue region.

In an embodiment of the invention, the transducers comprise electrodes for exciting them to generate the USW pattern and comprising electrifying same electrodes on the transducers to generate the USW pattern and simultaneously to generate an electric field in the region overlaying the USW pattern.

There is further provided in accordance with an embodiment of the invention, a method of treating a tissue region of a patient, the method comprising: coupling at least two ultrasound transducers having electrodes to the patient's skin; and electrifying electrodes on the transducers that excite the transducers to generate a pattern of ultrasound in the tissue region wherein voltage on the electrified electrodes simultaneously generates an electric field that overlays the ultrasound pattern in the region. Additionally or alternatively, the electric field is optionally an RF field.

In an embodiment of the invention, the method comprises: mounting the ultrasound transducers in a vessel; drawing up the tissue region into the vessel using a vacuum; and distorting the drawn up tissue region to improve coupling of the region to the transducers.

There is further provided in accordance with an embodiment of the invention, a method of coupling a tissue region underlying a region of skin to an ultrasound transducer, the method comprising: providing a vessel comprising an ultrasound transducer; drawing up the tissue region into the vessel using a vacuum; and distorting the drawn up tissue region to increase an area of the skin interfaced with the transducer. Additionally or alternatively, distorting optionally comprises applying mechanical force to the region. Optionally, applying force comprises applying force to the tissue region mechanically.

In an embodiment of the invention, the method comprises periodically distorting the tissue region. Optionally, periodically distorting comprises distorting at a resonant frequency of the tissue region. Additionally or alternatively, periodically distorting optionally comprises distorting at a frequency determined by a relaxation time of the tissue region.

There is further provided in accordance with an embodiment of the invention, apparatus for treating a tissue region underlying skin with ultrasound comprising: at least one transducer that is coupled to the skin; a power supply configured to excite the at least one transducer to generate a first ultrasound standing wave (USW) pattern at a first frequency in the tissue region and simultaneously generate a second USW pattern in the region at a second frequency different from the first frequency that spatially overlaps the first USW pattern. Optionally, the first and second patterns are substantially mutually completely overlapping.

Additionally or alternatively, the at least one ultrasound transducer optionally comprises two ultrasound transducers that face each other across the tissue region. Optionally, the first and second frequencies are resonant frequencies determined by the speed of sound in the tissue region and distance between the transducers. Additionally or alternatively, the transducers optionally have resonant frequencies of vibration substantially equal to the first and second resonant frequencies.

In an embodiment of the invention, the apparatus comprises a layer of material sandwiched between a transducer of the at least one transducer and the skin that positions the USWs so that pressure amplitude of the USWs along the skin is relatively small.

There is further provided in accordance with an embodiment of the invention, apparatus for treating a tissue region underlying skin with ultrasound, the apparatus comprising: an ultrasound transducer for transmitting ultrasound through the skin to generate an ultrasound standing wave (USW) in the tissue region; and a layer of material between the transducer and the skin that positions the USWs so that pressure amplitude of the USWs along the skin is relatively small. Additionally or alternatively, the layer optionally has acoustic impedance substantially equal to that of the tissue region.

In an embodiment of the invention, thickness of the layer is substantially equal to a quarter wavelength of ultrasound that the transducer transmits in the tissue region.

In an embodiment of the invention, the transducers comprise electrodes for exciting them to generate the USW patterns and comprising a power supply that electrifies electrodes on the transducers to generate the USW patterns, wherein the electrified electrodes simultaneously generate an electric field that overlays the USW pattern in the region.

There is further provided in accordance with an embodiment of the invention, a apparatus for treating a tissue region underlying skin with ultrasound, the apparatus comprising: at least two ultrasound transducers having electrodes for exciting the transducers to generate ultrasound in the tissue region; and a power supply that electrifies the electrodes to excite the transducers to generate a pattern of ultrasound in the tissue region wherein the electrified electrodes simultaneously generate an electric field that overlays the USW pattern in the region. Additionally or alternatively, the electric field is optionally an RF field.

In an embodiment of the invention, the apparatus comprises: a vessel comprising the at least one ultrasound transducer; a vacuum pump that generates vacuum in the vessel for drawing up the tissue region into the vessel; and an element that protrudes into the vessel and distorts the drawn up tissue region to improve coupling of the region to the at least one transducer.

There is further provided in accordance with an embodiment of the invention, a apparatus for treating a tissue region underlying skin with ultrasound, the apparatus comprising: a vessel comprising the at least one ultrasound transducer; a vacuum pump that generates vacuum in the vessel for drawing up the tissue region into the vessel; and an element that protrudes into the vessel and distorts the drawn up tissue region to improve coupling of the region to the at least one transducer. Additionally or alternatively, the element is optionally controllable to generate a time varying distortion to the tissue region. Optionally, the time varying distortion comprises a periodic distortion at a resonant frequency of the tissue region. Additionally or alternatively, the time varying distortion optionally comprises a periodic distortion at a frequency determined by a relaxation time of the tissue region.

### BRIEF DESCRIPTION OF THE DRAWINGS

Non-limiting examples of embodiments of the invention are described below with reference to figures attached hereto that are listed following this paragraph. Identical structures, elements or parts that appear in more than one figure are generally labeled with a same numeral in all the figures in which they appear. Dimensions of components and features shown in the figures are chosen for convenience and clarity of presentation and are not necessarily shown to scale.

FIG. 1 schematically shows a cross section of a perspective view of an ultrasound apparatus comprising a vacuum vessel for positioning a region of tissue for treatment with ultrasound in accordance with prior art;

FIG. 2 schematically shows an ultrasound treatment apparatus (UTA) for treating a region of tissue comprising a vacuum vessel for holding and positioning the skin for treatment with ultrasound, and having a shape adapter for distorting the tissue region, in accordance with an embodiment of the invention; and

FIG. 3 schematically shows a UTA similar to that shown in FIG. 2 comprising a configuration of ultrasound transducers for generating ultrasound standing waves (USW) in a region of tissue treated with the apparatus, in accordance with an embodiment of the invention.

### DETAILED DESCRIPTION

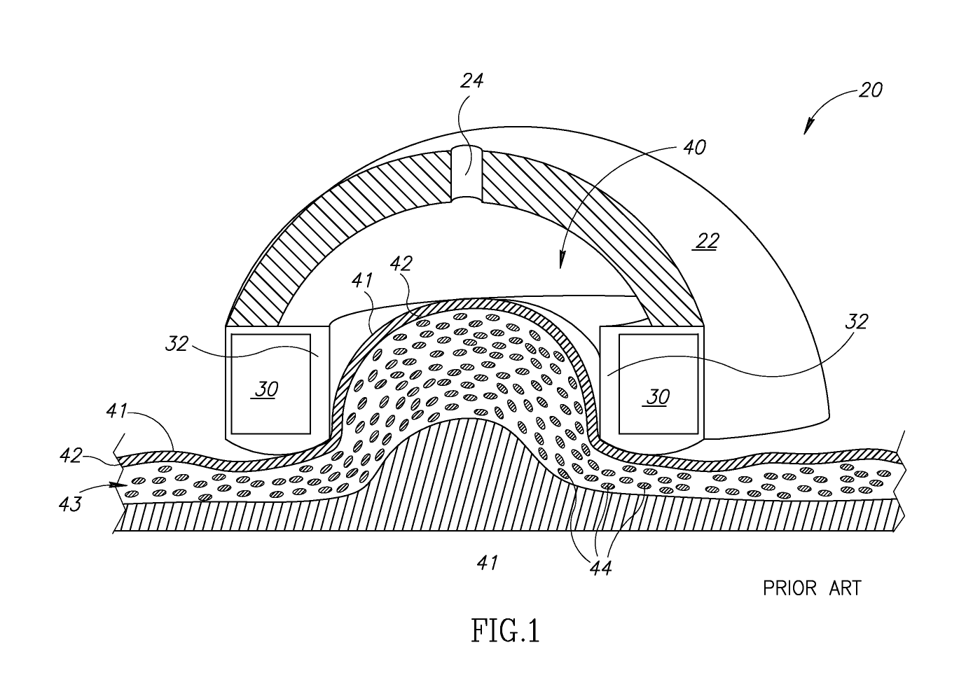

FIG. 1 schematically shows a cross section of a perspective view of an ultrasound apparatus 20 for treating a patient with ultrasound, optionally to lyse and remove subcutaneous adipose tissue from a tissue region of the patient, in accordance with prior art. In FIG. 1 apparatus 20 is shown treating a patient's tissue region 40.

Apparatus 20 comprises a vacuum vessel 22 formed having an outlet orifice 24 and comprising an array of ultrasound transducers 30 and a suitable power supply (not shown) to drive the transducers. By way of example, vacuum vessel 22 is an elongated, cup shaped vessel comprising two ultrasound transducers 30 that are located along a bottom edge 23 of vessel 22 and face each other. Optionally, transducers 30 are substantially planar and parallel. Transducers 30 are generally covered with a matching layer 32, an acoustic coupler 32, of material having thickness equal to about a quarter of a wavelength of ultrasound generated by transducers 30 to reduce acoustic mismatch between transducers 30 and skin 40. Typically, impedance of matching layer 32 is equal to about the square root of the product of the impedances of transducer 30 and skin tissue region 40.

In operation, air is aspirated from vacuum vessel through outlet orifice 24 to draw a tissue region 40 of the patient's surface tissue, comprising epidermis 41 dermis 42 and a subcutaneous layer 43 having adipose tissue 44, and position the region between ultrasound transducers 30 so that adipose tissue 44 can be treated with ultrasound. By drawing tissue region 40 up and away from the patient's body, subcutaneous layer 43 is positioned so that a relatively large portion of adipose tissue 44 is located between transducers 30 in a layer substantially parallel to the normal position of the patient's skin on the body before the region 40 is drawn up into vessel. Transducers 30 are therefore controllable to transmit ultrasound for treating adipose tissue 44 along directions that do not enter the body and possibly deleteriously affect the patient's internal organs.

However, because of normal elasticity of the skin and underlying regions of tissue region 40, tissue region 40 drawn up into vessel 22 does not make continuous, intimate contact with acoustic couplers 32, and acoustic contact between transducers 30 and the skin drawn into the vessel is generally incomplete. As a result, efficacy of treatment of adipose tissue 44 with ultrasound generated by the transducers compromised. FIG. 1 schematically shows a shape, reminiscent of a Gaussian surface, indicative of how tissue region 40 is shaped when drawn up into vessel 22 and incomplete contact of epidermis 41 with matching layers 32.

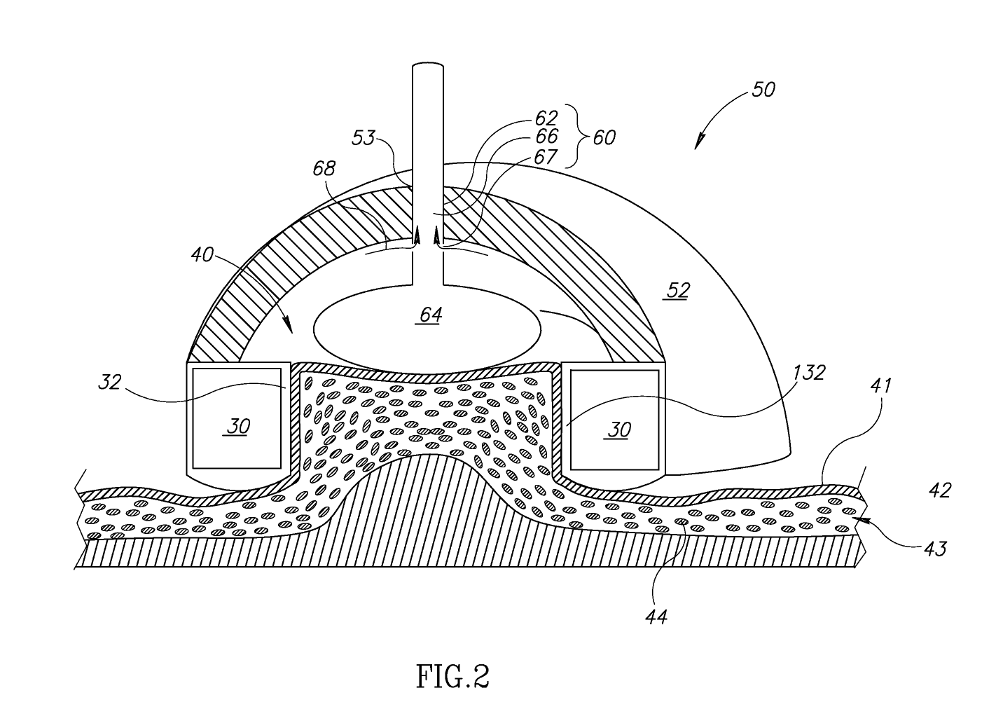

FIG. 2 schematically shows a cross section of a perspective view of an ultrasound treatment apparatus 50 (UTA) in accordance with an embodiment of the invention. UTA 50 comprises a vacuum vessel 52 for drawing up and holding a region of tissue region 40 to be treated with ultrasound, and an array of ultrasound transducers 30 for generating the ultrasound.

In an embodiment of the invention, transducers 30 are interfaced to tissue region 40 by protective buffer layers 132. Protective buffers 132 have thickness equal to about a quarter wavelength of ultrasound generated by transducers 30 and acoustic impedance substantially equal to about that of tissue 40. Operation of the protective buffers is discussed below. Optionally, vacuum vessel 52 has a shape similar to that of vacuum vessel 22 comprised in prior art apparatus 20, and ultrasound transducers 30 are configured and disposed similarly to those shown for the prior art apparatus.

However, UTA 50 comprises a device, i.e. a “shape adapter”, that distorts the shape of tissue drawn up into vacuum vessel 52 so that the drawn up tissue makes substantially continuous and intimate contact with buffers 132. In an embodiment of the invention, the shape adapter comprises a “plunger” 60 having a stem 62 that protrudes into vacuum vessel 52 through an aperture 53 and is mounted with a plunger head 64. Optionally, stem 62 comprises a pipe 66 formed having spiracles 67 through which air may be aspirated from vessel 52 to provide partial vacuum for drawing skin into the vessel. The pipe is sealed in aperture 53 to reduce, leakage of air between the pipe and walls of aperture so that a suitable partial vacuum for drawing up skin can be created in vessel 52. Arrows 68 schematically represent flow of air aspirated from vessel 52. Optionally, plunger head 64 is an oblate, “pumpkin shaped”, body. In some embodiments of the invention, the position of plunger 60 is fixed and located so that when tissue 40 is drawn up into vessel 52, plunger head 64 deforms the drawn up tissue, as shown in FIG. 2, so that it spreads laterally and makes close, continuous contact with buffers 132.

In some embodiments of the invention, plunger 60 is configured so that it is moveable “up” and “down” to adjust depth to which plunger head 64 intrudes into vessel 52. Optionally, plunger head 64 is moved up and down by moving stem 62 along its length in aperture 53. Sealing of stem 62 in aperture 53 to maintain suitable reduced leakage of air between the stem and aperture may be provided using any of various methods and materials known in the art. For example, stem 62 might be sealed against air leaks using a configuration of o-rings and/or vacuum greases.

It is noted that shape adapters in accordance with embodiments of the invention are not limited to plunger 60 and a shape adapter in accordance with an embodiment of the invention may be different from plunger 60. For example, a plunger type shape adapter may have a cylindrical or annular shaped plunger head. Alternatively, a plunger head may comprise a plurality of component elements such as an array of parallel cylinders that are pressed to skin drawn into vacuum vessel 52.

In some embodiments of the invention, plunger head 64 is formed from an elastic membrane and displacement of the plunger head is accomplished by filling the plunger head with a gas or liquid to expand it, or by removing gas or liquid to cause the plunger head to contract.

In some embodiments of the invention, plunger head 64 is repetitively moved during illumination of tissue region 40 with ultrasound to change force applied by the plunger head on the tissue region and thereby change position of mass points in the region relative ultrasound transmitted by transducers 30. In some embodiments of the invention, to change position of mass points in the region, vacuum vessel 52 is perturbated at a suitable frequency to generate repeated change in position of the mass points. The spatial shifting of mass points in tissue region 40, aids in homogenizing the effects of the ultrasound in the tissue region. Optionally, repetitive motion of the plunger head 64 is performed at a frequency substantially equal to a mechanical relaxation time and/or resonant frequency of tissue region 40. Mechanical resonant and relaxation time frequencies of skin tissue range from about 300 Hz to about 10 kHz. By mechanically vibrating tissue region 40 at a resonant frequency, motion of mass points in the tissue region tend to be relatively large and effects, such as tissue heating, of the motion amplified. By mechanically vibrating tissue region 40 at a relaxation time frequency, cavitation effects in the tissue region are amplified.

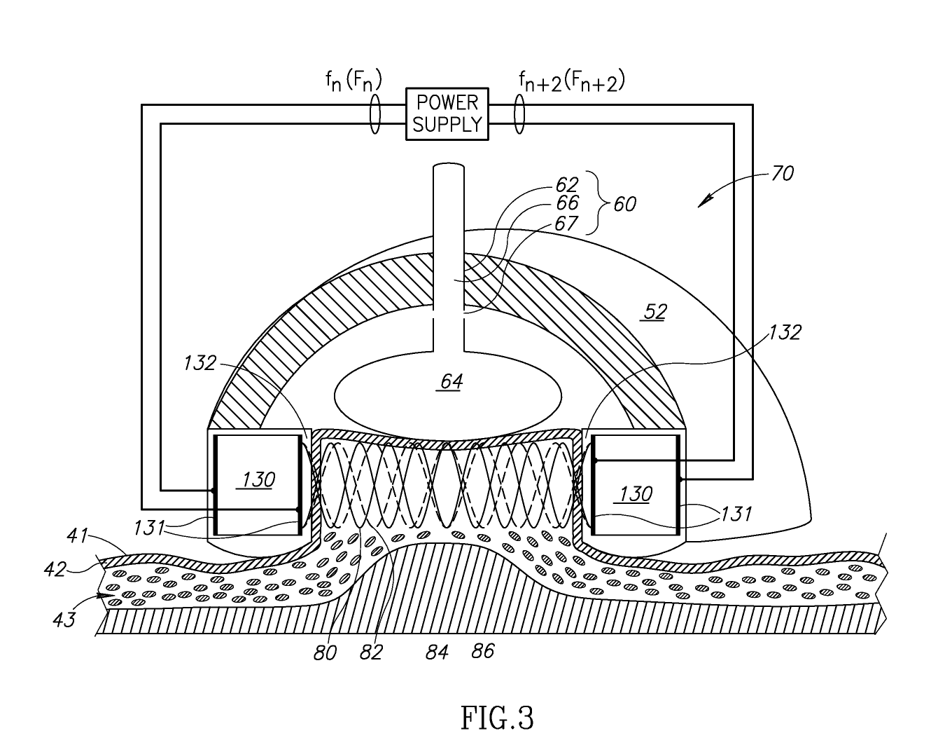

In accordance with some embodiments of the invention, a UTA comprises ultrasound transducers configured to simultaneously superpose at least two ultrasound standing wave (USW) patterns of at different frequencies in a region of tissue. FIG. 3 schematically shows a UTA 70 configured to superpose at least two USW patterns in a region of tissue being treated by the UTA in accordance with an embodiment of the invention.

UTA 70 optionally comprises a vacuum vessel 52 for drawing up and positioning a tissue region 40 between ultrasound transducers 130 and a shape adapter 60 for providing improved acoustic coupling of the tissue region to the transducers, in accordance with an embodiment of the invention. Transducers 130 are optionally interfaced with tissue region 40 by buffers 132. Optionally, the vacuum vessel, shape adapter and transducers are similar to those comprised in UTA 50 shown in FIG. 2. In accordance with an embodiment of the invention, UTA 70 comprises a power supply 72 controllable to excite transducers 130 to simultaneously generate at least two USW patterns that overlap to superpose in region 40 positioned between the transducers. Power supply 72 is schematically shown coupled to electrodes 131 that are electrified by the power supply to excited transducers 130 and providing by way of example, as discussed below AC power at two frequencies fₙ and fₙ₊₂. Whereas in the figure it appears that power supply 72 supplies one set of electrodes 131 at frequency fₙ and the other set at frequency fₙ₊₂, generally both frequencies are provided to both sets of electrodes simultaneously by electrifying the electrodes with a suitably amplitude modulated AC voltage. In some embodiments of the invention the AC power is supplied in pulses.

According to an embodiment of the invention, power supply 72 controls transducers 130 to generate two standing wave patterns at, optionally, relatively close resonant frequencies. The inventor has determined that superposing at least two standing wave patterns at relatively close resonant frequencies in a tissue region generates a relatively dense pattern of nodes and antinodes that cyclically shift their location in the tissue at a relatively low frequency equal to about one half a difference in the frequencies of the two USW patterns. The cyclical spatial motion of the nodes and antinodes homogenizes the effects of the ultrasound on the tissue. Operating at resonant frequencies provides for buildup of relatively intense USW patterns in the tissue. It is noted that unlike in prior art methods of using standing wave patterns of ultrasound in which the patterns are generated off-resonance to protect the skin, the use of buffers 132 in accordance with an embodiment of the invention enables skin safe generation of USW patterns on-resonance.

In some embodiments of the invention, a difference in the frequencies of the two USW patterns is determined responsive to a relaxation time or a resonant frequency of tissue components to enhance effects of the USW pattern in the tissue. For example, the frequencies are optionally determined so that rate of decrease, characterized by a tissue relaxation time, in a number of micro-bubbles formed in the tissue by one of the USW, is slowed by action of the other USW pattern. The dual effect of both USW patterns in accordance with an embodiment of the invention enhances cavitation in the tissue.

Establishing a standing wave pattern between two boundaries a given distance apart requires that wavelengths of traveling waves that establish the standing waves satisfy a constraint that the given distance is an integer multiple, “n”, of a half wavelength of the traveling waves. Let distance between transducers 130 be represented by D. Let the wavelength of the traveling ultrasound waves generated by the transducers to create standing waves in tissue region 40 be represented by “λₙ”. Then the constraint requires that D=nλₙ/2. If the speed of sound in tissue is represented by “c”, the constraint may be written in terms of frequency “fₙ” of the traveling waves as fₙ=nc/2D.

Each transducer 30 operates substantially as a rigid reflector of acoustic waves incident thereon. As a result, the displacement phasor (conventionally the “s” phasor or wave) of a reflection of an acoustic wave incident on a transducer 30 is, at the transducer face, 180° out of phase with the displacement phasor of the incident of the wave at the face, and the pressure phasor of the reflected wave is in phase with that of the incident wave. Therefore, for a standing wave pattern generated by transducers 130 in tissue region 40, the pattern has a pressure antinode at each transducer 30 and a pressure node at a location displaced by a distance equal to about ¼λ from the transducer. Protective buffers 132, in accordance with an embodiment of the invention, operate to displace the pressure antinode at a transducer 30 from skin 41 of tissue region 40 and position the “λ/4 pressure node” at the skin surface. The protective buffers protect thereby the skin from damage by large magnitude pressures that generally develop in the USW pattern. It is noted that for odd numbered frequencies fₙ if a protective buffer is configured to position a node at the skin surface for f₁ it will also position a node at the skin surface for all other odd numbered frequencies. For convenience of presentation, when referring to phase or magnitude of an acoustic wave, unless otherwise specified, the referral is to the pressure phasor of the wave and not the displacement phasor.

Because of the phase reversal of the displacement phasor at a transducer 30, to establish and maintain standing acoustic waves between transducers 30, in accordance with an embodiment of the invention, for n odd in the expression D=nλₙ/2, transducers 130 are driven in phase by power supply 72 and for n even, the transducers are driven 180° out of phase. And, in accordance with an embodiment of the invention, to establish two standing wave patterns at different frequencies if one of the frequencies is fₙ=nc/2D, the other frequency is fₙ₊₂=(n+m2)c/2D, where m is an integer. Optionally, m=1.

In some embodiments of the invention, natural resonant frequencies of transducers 130 are matched to substantially coincide with resonance frequencies of the system comprising the transducers and tissue region 40. For example, transducers 130 are generally excited at odd numbered harmonics, e.g. 1st, 3rd, 5th, etc harmonic vibration frequencies of the transducers to generate ultrasound. Typically 1st and 3rd harmonics are excited. In an embodiment of the invention the transducers are configured so that they substantially coincide with the resonant frequencies of the tissue region and spacing D between the transducers.

In FIG. 3, transducers 130 and power supply 72 are indicated configured to generate standing waves in tissue 40 for frequencies fₙ=nc/2D and fₙ₊₁=(n+2)c/2D. By way of example in the figure fₙ=f₈ and fₙ₊₁=f₁₀. Standing waves generated by transducers 130 at frequencies f₈ and f₁₀ are schematically represented by solid and dashed lines 80 and 82 respectively that represent pressure of the waves. Location of pressure antinodes of standing waves 80 and 82 are schematically indicated by open and closed circles 84 and 86 respectively. It is noted that the pressure nodes closest to transducers 130 are located substantially at the surface of skin 41, thereby protecting the skin from “pressure burning”.

Optionally, to generate standing waves 80 and 82 power supply 72 excites a first one of transducers 130 to transmit ultrasound waves at frequency fₙ, and a second one of transducers 30 to transmit ultrasound waves at frequency fₙ₊₂. Reflections of the transmitted ultrasound waves from the second and first transducers 130 respectively combine with the transmitted waves to generate standing waves 80 and 82. In some embodiments of the invention, each transducer 30 is excited to transmit ultrasound waves toward the other of transducers 130 at both frequencies fₙ and fₙ₊₂. The transmitted waves propagating in opposite directions combine to generate standing waves 80 and 82.

Superposed standing waves 80 and 82 provide a shifting “USW” pattern of pressure nodes and antinodes that may be described by an expression:

A(x,t)=2A cos(kₒx)cos(Δkx/2)cos(A ωt/2)cos(ωₒt)+2A sin(kₒx)sin(Δkx/2)sin(Δωt/2)sin(ωₒt). (1)

In the above equation, A(x,t) is ultrasound amplitude at a coordinate x between transducers 130 at time t and kₒ=(π/λₙ+π/λₙ₊₁), Δk=|(π/λₙ−π/λₙ₊₁)|, ωₒ=(πfₙ+πfₙ₊₁), and Δω=|(πfₙ−πfₙ₊₁)|. The growth of amplitude A(x,t) at a resonance of tissue region 40 and transducers 130 is not indicated by expression (1). It is expected that increase in acoustic energy stored in the USW pattern in tissue region 40 generated by excitation of transducers 130 will result in A(x,t) having a maximum substantially equal to 4QA, where Q is a quality factor characterized by the rate of dissipation of acoustic energy in the tissue and transducers 30. For adipose tissue and an ultrasound treatment apparatus, UTA, in accordance with an embodiment of the invention similar to UTA 70, the quality factor Q can have a relatively large value between 10-20.

From equation (1) and FIG. 3 it is seen that superposed standing waves 80 and 82 generate a relatively dense pattern of standing ultrasound waves in tissue region 40 between transducers 130 having interleaved nodes and antinodes Ultrasound amplitude A(x,t) at adjacent antinodes slowly cycle harmonically at a frequency Δω/2, and out of phase by 90°, between substantially zero and maxima determined by the value of spatial “envelope” functions cos(Δkx) and sin(Δkx). The overlapping standing wave patterns 80 and 82 therefore superpose to generate a pattern of ultrasound node and antinodes that cyclically shift their location in the tissue at a relatively low frequency, i.e. Δω, equal to about a difference in the frequencies of the two USW patterns. The cyclical spatial motion of the nodes and antinodes homogenizes the effects of the ultrasound on the tissue.

By way of numerical example, let D, the distance between transducers 130 be 30 mm and let n=8 and n+1=10. The speed of sound in adipose tissue is about 1,500 m/s. Then fₙ=200 kHz, λₙ=about 7.5 mm, fₙ₊₁=250 kHz and λₙ=6 mm. Antinodes and nodes of standing waves 80 and 82 cycle harmonically at a frequency (Δω/2 (see expression 1)) of about 25 kHz. And spatial envelope functions have a spatial frequency (Δk/2 equal to about π/60 mm⁻¹ and a spatial period equal to about 19 mm. It is noted that numerical values, and details of FIG. 3 are schematic and provided for idealized configurations and that in practice the values given and acoustic patterns shown may differ from those presented.

In some embodiments of the invention, as is by way of example shown for FIG. 3, frequencies fₙ and fₙ₊₂ have n even. For such frequencies, in accordance with an embodiment of the invention, transducers 130 are driven 180° out of phase. As a result, transducer electrodes 131 that are opposite and face each across tissue region 40 have opposite polarity and generate an AC filed in the tissue region at a same frequency as AC power used to excite transducers 130. In an embodiment of the invention, power supply 72 and transducers 130 are configured so that the frequencies and amplitudes of the voltages used to drive transducers 130 provide a desired RF field in the tissue region. As noted above, because the same electrodes are used to excite transducers 130 and generate the RF field the RF field substantially overlaps and superpose on an ultrasound pattern generated by transducers 130 in the tissue. Synergistic treatment of tissue region 40 by the RF and acoustic fields is thereby promoted.

For example, an RF field pattern preferentially heats relatively high resistance portions of tissue region 40. If an ultrasound field generated by transducers 130 is configured to lyse adipose tissue 44 in subcutaneous layer 43 of tissue region 40 by cavitation, resistance of cavitated portions increases relative to surrounding, non-cavitated tissue. As a result, the RF field preferentially heats the cavitated adipose tissue region 44, amplifying and destructive effects of the ultrasound field on the adipose tissue and tending to localize the effects to the adipose tissue. The ultrasound thereby tends to focus and localize the RF field effects to the portions to be lysed and provides better discrimination of therapy provided by UTA 70.

It is noted, that electrodes 131 facing each other across tissue region 40 and the tissue region present a complex, mainly capacitive, electrical impedance to a voltage generated between the electrodes. In accordance with an embodiment of the invention, changes in complex impedance are monitored using voltage applied between the electrodes. The changes in complex impedance are optionally used to monitor and control changes in tissue region 40 that result from treatment of the tissue region by fields created by UTA 70. It is further noted that using USW simultaneously at a plurality frequencies to treat a tissue region in accordance with an embodiment of the invention is relatively self-limiting. Upon generating substantial heating and/or cavitation in the tissue region, the USW tends to “self extinguish”. The heating and/or cavitation causes acoustic attenuation in the tissue region to increase to such an extent that it strongly damps acoustic traveling waves used to generate the USW. The heating and/or cavitation also causes change in the velocity of sound in the tissue region. Change in the velocity of sound in turn causes the system of tissue and transducers to go off resonance and amplitude of USW in the region to substantially decrease.

Whereas the application of USW and RF filed to tissue have been described using by way of example, a UTA having parallel planar acoustic transducers and electrodes on the transducers, the present invention is not limited to configurations having parallel planar transducers. For example, a UTA in accordance with an embodiment of the invention may have a cylindrical shape and optionally a single cylindrical transducer powered to generate overlapping USW in a tissue region. For such a configuration, however, frequencies of the waves are limited to frequencies fₙ for which “n” is odd. For frequencies for which n is even, a plurality of independently driven transducers rather than a single transducer may be used.

In the description and claims of the present application, each of the verbs, “comprise” “include” and “have”, and conjugates thereof, are used to indicate that the object or objects of the verb are not necessarily an exhaustive listing of members, components, elements or parts of the subject or subjects of the verb.

The invention has been described with reference to embodiments thereof that are provided by way of example and are not intended to limit the scope of the invention. The described embodiments comprise different features, not all of which are required in all embodiments of the invention. Some embodiments of the invention utilize only some of the features or possible combinations of the features. Variations of embodiments of the described invention and embodiments of the invention comprising different combinations of features than those noted in the described embodiments will occur to persons of the art. The scope of the invention is limited only by the following claims.

1-36. (canceled)

37. An apparatus for treating a region of tissue underlying skin with ultrasound waves which enter the tissue through a skin surface, the tissue characterized by a tissue acoustic impedance, the apparatus comprising:
a) at least one ultrasound transducer;
b) a power supply configured to simultaneously excite the at least one ultrasound transducer to generate a first ultrasound standing wave (USW) pattern of nodes and antinodes at a first frequency f₁ and a second USW pattern of nodes and antinodes at a second frequency f₂ different from f₁, wherein the first and second USW patterns fully overlap spatially in the tissue region; and
c) a protective buffer layer interfacing between the at least one ultrasound transducer and the skin, the protective buffer layer having a thickness equal to a quarter wavelength of ultrasound generated by the at least one ultrasound transducer and an acoustic impedance substantially equal to the tissue acoustic impedance, the protective buffer layer used for positioning USW pressure nodes along the skin surface, thereby reducing damage to the skin surface.

38. The apparatus of claim 37, wherein the at least one ultrasound transducer includes a single, substantially cylindrical ultrasound transducer with a diameter D.

39. The apparatus of claim 37, wherein the at least one ultrasound transducer includes two ultrasound transducers that face each other across the tissue region and are separated by a spacing D, a first ultrasound transducer for generating the first USW pattern at the first frequency f₁ and a second ultrasound transducer for generating the second pattern at the second frequency f₂.

40. The apparatus of claim 37, wherein f₁ and f₂ are resonant frequencies determined by the speed of sound in the tissue region and by an internal transducer spacing or diameter D.

41. The apparatus of claim 38, further comprising:
d) a vessel comprising the single, substantially cylindrical ultrasound transducer;
e) a vacuum pump that generates vacuum in the vessel for drawing up the tissue region into the vessel; and
f) a shape adapter element that distorts the drawn up tissue region to couple the tissue region to the single ultrasound transducer.

42. The apparatus of claim 41, wherein the shape adapter element includes a protruding element that protrudes into the vessel and is configured to change a position of mass points in the tissue region relative to the generated USW patterns.

43. The apparatus of claim 39, further comprising:
d) a vessel comprising the two ultrasound transducers;
e) a vacuum pump that generates vacuum in the vessel for drawing up the tissue region into the vessel; and
f) a shape adapter element that distorts the drawn up tissue region to improve coupling of the tissue region to each ultrasound transducer.

44. The apparatus of claim 43, wherein the shape adapter element includes a protruding element that protrudes into the vessel and is configured to change a position of mass points in the tissue region relative to the generated USW patterns.

45. The apparatus of claim 44, wherein the protective buffer layer is electrically conductive and wherein each ultrasound transducer includes electrodes electrified by the power supply, the power supply further operative to generate an electric field that overlays the USW patterns in the tissue region simultaneously with the generation of the USW patterns.

46. The apparatus of claim 45, wherein the electric field is a radio frequency field.

47. A method for treating a region of tissue underlying skin with ultrasound waves which enter the tissue through a skin surface, the tissue characterized by a tissue acoustic impedance, the method comprising the steps of:
a) simultaneously generating in the tissue region, using at least one ultrasound transducer, a first ultrasound standing wave (USW) pattern at a first frequency f₁ and a second USW pattern at a second frequency f₂ different from f₁, wherein each of the USW patterns includes pressure nodes and antinodes and wherein the two USW patterns fully overlap spatially in the tissue region; and
b) using a protective buffer layer having a thickness equal to a quarter wavelength of ultrasound generated by the at least one ultrasound transducer and an acoustic impedance substantially equal to the tissue acoustic impedance to position USW pressure nodes along the skin surface, thereby reducing damage to the skin surface.

48. The method of claim 47, wherein the step of simultaneously generating includes using a single, substantially cylindrical ultrasound transducer with a diameter D for generating the USW patterns at the first and second frequencies.

49. The method of claim 47, wherein the step of simultaneously generating includes using two ultrasound transducers that face each other across the tissue region and are separated by a spacing D, a first ultrasound transducer for generating the first USW pattern at the first frequency and a second ultrasound transducer for generating the second pattern at the second frequency.

50. The method of claim 47, wherein f₁ and f₂ are resonant frequencies determined by the speed of sound in the tissue region and by an internal transducer spacing or diameter D.

51. The method of claim 48, further comprising the steps of:
d) providing a vessel comprising the single, substantially cylindrical ultrasound transducer;
e) drawing up the tissue region into the vessel by applying vacuum to the vessel; and
f) distorting the drawn up tissue region to couple the tissue region to the ultrasound transducer.

52. The method of claim 51, wherein the distorting is done using a shape adapter element which includes a protruding element that protrudes into the vessel and is configured to change a position of mass points in the tissue region relative to the generated USW patterns.

53. The method of claim 49, further comprising the steps of:
d) providing a vessel comprising the two ultrasound transducers;
e) drawing up the tissue region into the vessel by applying vacuum to the vessel; and
f) distorting the drawn up tissue region to couple the tissue region to the two ultrasound transducers.

54. The method of claim 53, wherein the distorting is done using a shape adapter element which includes a protruding element that protrudes into the vessel and is configured to change a position of mass points in the tissue region relative to the generated USW patterns.

55. The method of claim 53, wherein the protective buffer layer is electrically conductive and wherein the two ultrasound transducers include electrodes electrified by the power supply, the power supply further operative to generate an electric field that overlays the USW patterns in the tissue region simultaneously with the generation of the USW patterns.

56. The method of claim 55, wherein the electric field is a radio frequency field.

57. A method for treating a region of tissue underlying skin with ultrasound waves which enter the tissue through a skin surface, the tissue characterized by a tissue acoustic impedance, the method comprising the steps of:
a) providing at least one ultrasound transducer; and
b) exciting the at least one ultrasound transducer to simultaneously generate in the tissue region a first ultrasound standing wave (USW) pattern at a first frequency f₁ and a second USW pattern at a second frequency f₂ different from f₁, wherein each of the USW patterns includes pressure nodes and antinodes, wherein the two USW patterns fully overlap spatially in the tissue region and wherein f₁ and f₂ are resonant frequencies determined by the speed of sound in the tissue region and by an internal transducer spacing or diameter D, the two resonant frequencies chosen such as to cause cyclical shifting of the spatially overlapped USW patterns over the tissue region at a frequency equal to about a difference between frequencies f₁ and f₂;
whereby the cyclically shifting USW patterns provide homogeneous tissue treatment.

58. The method of claim 57, further comprising the step of
c) using a protective buffer layer having a thickness equal to a quarter wavelength of ultrasound generated by the at least one ultrasound transducer and an acoustic impedance substantially equal to the tissue acoustic impedance to position USW pressure nodes along the skin surface, thereby reducing damage to the skin surface.
